(19) United States
(12) Patent Application Publication
(10) Pub. No.: US 2023/0146308 A1
(43) Pub. Date: May 11, 2023

(54) METHOD FOR THE TREATMENT NLRP3-ASSOCIATED DISEASES

(71) Applicant: Softhale NV, Diepenbeek (BE)

(72) Inventors: Juergen Rawert, Koeln (DE); Jan-Torsten Tews, Munich (DE)

(21) Appl. No.: 17/905,416

(22) PCT Filed: Mar. 17, 2021
(86) PCT No.: PCT/EP2021/056829

§ 371 Date: Sep. 1, 2022

Related U.S. Application Data
(60) Provisional application No. 62/991,937, filed on Mar. 19, 2020.

(51) Int. Cl. A61K 9/00; A61M 11/00; A61K 31/64; A61K 31/275; A61K 31/277; A61K 31/4025; A61K 31/343

(52) U.S. Cl. CPC A61K 9/0078; A61M 11/007; A61K 31/64; A61K 31/275; A61K 31/277; A61K 31/4025; A61K 31/343

(57) ABSTRACT

The present invention provides for a method for the treatment of a NLRP3-associated disease or condition in a subject, the method comprising the step of administering to said subject a medically active liquid in nebulized form by inhalation, wherein the medically active liquid comprises a NLRP3 inhibitor and wherein the medically active liquid is administered in nebulized form using an inhalation device.

### BACKGROUND OF THE INVENTION

The present invention relates to the field of methods for the treatment or prevention as well as compositions for use in the treatment or prevention of NLRP3-associated diseases, disorders or conditions, more specifically to the field of methods for the treatment of pulmonary NLRP3-associated diseases, disorders or conditions, such as inflammatory events, viral infections, specifically infections by coronavirus. Furthermore, the present invention relates to the field of inhalation devices or the administration of medically active liquids for inhalation therapy. More specifically, the present invention relates to the administration of a medically active liquid comprising a NLRP3 inhibitor by inhalation.

Nebulizers or other aerosol generators for liquids are known from the art. Amongst others, such devices are used in medical science and therapy. There, they serve as inhalation devices for the application of active ingredients in the form of aerosols, i.e., small liquid droplets embedded in a gas. Such an inhalation device is known e.g., from document EP 0 627 230 B1. Essential components of this inhalation device are a reservoir in which the liquid that is to be aerosolized is contained; a pumping device for generation of a pressure being sufficiently high for nebulizing; as well as an atomizing device in the form of a nozzle. By means of the pumping device, the liquid is drawn in a discrete amount, i.e., not continuously, from the reservoir, and fed to the nozzle. The pumping device works without a propellant and generates pressure mechanically.

Inflammasomes are large intracellular multiprotein complexes that play a central role in innate immunity. They detect and respond to a large range of pathogen-associated molecular patterns (PAMPs), including bacterial flagellin, and damage-associated molecular patterns (DAMPs), such as uric acid crystals. Inflammasomes contain a member of the NOD-like receptor (NLR) family, such as NLRP3 and IPAF, by which they are defined. The NLR protein recruits the inflammasome-adaptor protein ASC, which in turn interacts with caspase-1 leading to its activation. Once activated, caspase-1 promotes the maturation of the proinflammatory cytokines interleukin IL-1β and IL-18.

Furthermore, it has been reported that NOD-like receptor family, pyrin domain-containing 3 (NLRP3) is activated by a wide variety of stimuli, including virus infection and that high levels of proinflammatory cytokines, including tumor necrosis factor (TNF)-α interleukin IL-1β, and IL-6, were detected in autopsy tissues from SARS patients.

WO 2016/131098 A1 discloses sulfonylureas and related compounds which have advantageous properties and show useful activity in the inhibition of activation of the NLRP3 inflammasome and in the treatment of a wide range of disorders.

I.-Y. Chen et al. describe in Frontiers in Microbiology, January 2019, Vol. 10; Article 50 (doi: 10.3389/fmicb.2019.00050) that the Severe Acute Respiratory Syndrome (SARS) coronavirus viroporin 3a activates the NLRP3 inflammasome. The authors describe that NOD-like receptor family, pyrin domain-containing 3 (NLRP3) regulates the secretion of proinflammatory interleukin 1 beta (IL-10) and IL-18. The authors further provide evidence that SARS-CoV 3a protein activates the NLRP3 inflammasome in lipopolysaccharide-primed macrophages and that SARS-CoV 3a was sufficient to cause the NLRP3 inflammasome activation.

A. Zahid et al. describe in Frontiers in Immunology, October 2019, Vol. 10; Article 2538 (doi: 10.3389/fimmu.2019.02538) pharmacological inhibitors of the NLRP3 inflammasome. The authors report that recent investigations have disclosed various inhibitors of the NLRP3 inflammasome pathway which were validated through in vitro studies and in vivo experiments in animal models of NLRP3-associated disorders. Some of these inhibitors directly target the NLRP3 protein whereas some are aimed at other components and products of the inflammasome.

D. Bai et al. report in American Journal of Respiratory and Critical Care Medicine 2019; 199: A4605 the evaluation of a NLRP3 pulmonary delivery antisense strategy for the treatment of idiopathic pulmonary fibrosis (IPF). The authors report that antisense oligonucleotides (ASOs) were administered orotracheally to the lungs of mice twice a week. It was found that the NLRP3 ASOs were effective at reducing target mRNA in the bleomycin induced IPF models, and, furthermore, that NLRP3 ASOs were able to prevent bleomycin induced endpoints, including minimization of body weight loss and improved survival. It should be pointed out, however, that the antisense oligonucleotides were administered orotracheally, i.e., by a tube inserted through the mouth into the trachea.

It is thus an object of the present invention to provide a method for the effective prevention or treatment of NLRP3-associated disease or disorders especially in an effective and patient friendly manner. Further objects of the invention will be clear on the basis of the following description of the invention, examples and claims.

### SUMMARY OF THE INVENTION

In a first aspect, the invention relates to a medically active liquid comprising a NLRP3 inhibitor for use in the treatment or prevention of a NLRP3-associated disease, disorder or condition in a subject, wherein the medically active liquid is administered to the subject in nebulized form by inhalation using an inhalation device.

In a second aspect, the invention provides for a method for the treatment or prevention of a NLRP3-associated disease, disorder or condition in a subject, the method comprising the step of administering to said subject a medically active liquid in nebulized form by inhalation,

wherein the medically active liquid comprises a NLRP3 inhibitor and wherein the medically active liquid is administered in nebulized form using an inhalation device.

In a third aspect, the present invention provides for the use of a NLRP3 inhibitor for the preparation of a medically active liquid for the treatment of a NLRP3-associated disease, disorder or condition, wherein the medically active liquid is administered to a subject in nebulized form by inhalation using an inhalation device.

In a fourth aspect, the present invention provides for the use of a medically active liquid comprising a NLRP3 inhibitor for the prevention or treatment of a NLRP3-associated disease, disorder or condition, wherein the medically active liquid is used by inhalation of the medically active liquid in nebulized form, wherein the medically active liquid in nebulized form is generated by nebulization using an inhalation device.

In a fifth aspect, the present invention provides for the use of an inhalation device for the prevention or treatment of a NLRP3-associated disease, disorder or condition in a subject, wherein the medically active liquid is administered in nebulized form using the inhalation device and wherein the medically active liquid comprises a NLRP3 inhibitor.

In a sixth aspect, the present invention provides for a kit, specifically for a kit for the treatment or prevention of a NLRP3-associated disease, disorder or condition in a subject, the kit comprising
a medically active liquid comprising a NLRP3 inhibitor for the prevention or treatment of a NLRP3-associated disease, disorder or condition, wherein the medically active liquid is adapted to be administered to the subject in nebulized form by inhalation; and
an inhalation device, preferably a hand-held inhalation device, such as a soft-mist-inhaler.

In a seventh aspect, the present invention provides for the use of a medically active liquid comprising a NLRP3 inhibitor in the manufacture of a kit for the treatment of a NLRP3-associated disease, disorder or condition in a subject, the kit comprising
a medically active liquid comprising a NLRP3 inhibitor for the prevention or treatment of a NLRP3-associated disease, disorder or condition, wherein the medically active liquid is adapted to be administered to the subject in nebulized form by inhalation; and
an inhalation device, preferably a hand-held inhalation device, such as a soft-mist-inhaler.

### BRIEF DESCRIPTION OF THE DRAWINGS

FIG. 1 shows one of the preferred embodiments of an inhalation device useful for the nebulization of the medically active liquid according to the present invention; the preferred inhalation device is depicted schematically and not-to-scale;

FIG. 1 shows the situation prior to first use;

FIG. 2 shows an inhalation device similar to the one of FIG. 1, but without an outlet valve;

FIG. 3 shows the embodiment of FIG. 1 with a filled pumping chamber;

FIG. 4 shows the situation during the first actuation of the inhalation device of FIG. 1;

FIG. 5 shows the situation at the end of the first actuation; and

FIG. 6 shows the situation after re-filling the pumping chamber.

### DETAILED DESCRIPTION OF THE INVENTION

In a first aspect, the present invention provides for a medically active liquid comprising a NLRP3 inhibitor for use in the treatment or prevention of a NLRP3-associated disease, disorder or condition in a subject, wherein the medically active liquid is administered to the subject in nebulized form by inhalation using an inhalation device.

Introductorily, some definitions of terms are given which are used throughout the description and claims. The definitions should be used to determine the meaning of the respective expressions unless the context requires a different meaning.

The term “about” or the like in connection with an attribute or value includes the exact attribute or precise value, as well as any attribute or value typically considered to fall within the normal or accepted variability associated with the technical field, and methods of measuring or determining said attribute or value.

“Atomization” and “nebulization” in the context of inhalers means the generation of fine, inhalable droplets of a liquid. The typical dimensions of atomized droplets are in the range of several microns.

An “aerosol” is a dispersion of a solid or liquid phase in a gas phase. The dispersed phase, also termed the discontinuous phase, is comprised of multiple solid or liquid particles. The aerosol generated by the inhalation device of the invention is a dispersion of a liquid phase in the form of inhalable liquid droplets in a gas phase which is typically air. The dispersed liquid phase may optionally comprise solid particles dispersed in the liquid.

The term “comprising,” and related terms “comprise” or “comprises” would be understood as meaning that features additional to the features prefaced by the term may be present. Conversely, the term “consists,” and related terms would be understood as meaning that no other features, other than those prefaced by the term are present, and if present, only in trace or residual amounts such as to confer no technical advantage or relevance in respect of the object of the invention.

The term “treatment” as used herein, means administration of the compound or composition to a subject to at least ameliorate, reduce or suppress existing signs or symptoms of the disease, disorder or condition experienced by the subject.

The term “prevention” as used herein means prophylactically administering the formulation to a subject who does not exhibit signs or symptoms of a disease disorder or condition, but who is expected or anticipated to likely exhibit such signs or symptoms in the absence of prevention. Preventative treatment may at least lessen or partly ameliorate expected symptoms or signs.

The term “effective amount” as used herein refers to the administration of an amount of the relevant compound or composition sufficient to prevent the occurrence of symptoms of the condition being treated, or to bring about a halt in the worsening of symptoms or to treat and alleviate or at least reduce the severity of the symptoms. The effective amount will vary in a manner which would be understood by a person of skill in the art with patient age, sex, weight etc.

The terms “subject” or “individual” or “patient” as used herein may refer to any subject, particularly a vertebrate subject, and even more particularly a mammalian subject, for whom therapy is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject, individual or patient is a human.

The term “medically active liquid” as used herein means a pharmaceutically acceptable liquid comprising at least one medically active compound.

As used herein, the term “medically active” refers to a compound which has pharmacologically activity which improves symptoms associated with and/or caused by a NLRP3-associated disease, disorder or condition.

Further definitions are provided in the subsequent description.

For the avoidance of doubt, it should be noted that all embodiments and features of the present invention as well as combinations thereof as described below regardless of being referred to as “specific”, “particular”, “preferred”, “advantageous” or in any other way may refer to all aspects of the present invention as summarized above and as additionally described below.

The present invention provides for compounds for use, uses as well as methods for the treatment or prevention of a NLRP3-associated disease, disorder or condition, or in other words, a disease, disorder or condition which is associated to, caused by or mediated through the NOD-like receptor family, pyrin domain-containing 3 (NLRP3). In specific embodiments, the NLRP3-associated disease, disorder or condition is one which is responsive to inhibition of activation of the NLRP3 inflammasome.

In general terms, a NLRP3-associated disease, disorder or condition may be a disease, disorder or condition of the immune system, the cardiovascular system, the endocrine system, the gastro-intestinal tract, the renal system, the respiratory system, the central nervous system, may be a cancer or other malignancy and/or may be caused by or associated with a pathogen.

More specifically, a NLRP3-associated disease, disorder or condition as referred to herein may be a disease, disorder or condition of the immune system, an inflammatory disease, disorder or condition or an autoimmune disease, disorder or condition, a disease, disorder or condition of the cardiovascular system, a cancer, tumor or other malignancy, a disease, disorder or condition of the renal system, a disease, disorder or condition of the gastro-intestinal tract, a disease, disorder or condition of the respiratory system, a disease disorder or condition of the endocrine system and/or a disease, disorder or condition of the central nervous system (CNS).

Exemplary NLRP3-associated diseases, disorders or conditions that may be treated or prevented according to the present invention comprise, but are not limited to constitutive inflammation including the cryopyrin-associated periodic syndromes (CAPS): Muckle-Wells syndrome (MWS), familial cold autoinflammatory syndrome (FCAS) and neonatal-onset multisystem inflammatory disease (NOMID); including autoinflammatory diseases: familial Mediterranean fever (FMF), TNF receptor associated periodic syndrome (TRAPS), mevalonate kinase deficiency (MKD), hyperimmunoglobulinemia D and periodic fever syndrome (HIDS), deficiency of interleukin 1 receptor (DIRA) antagonist, Majeed syndrome, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), sideroblastic anemia with B-cell immunodeficiency, periodic fevers, and developmental delay (SIFD); Sweet's syndrome, chronic nonbacterial osteomyelitis (CNO), chronic recurrent multifocal osteomyelitis (CRMO) and synovitis, acne, pustulosis, hyperostosis, osteitis syndrome (SAPHO); autoimmune diseases including multiple sclerosis (MS), type-1 diabetes, psoriasis, rheumatoid arthritis, Behcet's disease, Sjogren's syndrome and Schnitzler syndrome; respiratory diseases including chronic obstructive pulmonary disorder (COPD), steroid-resistant asthma, asbestosis, silicosis and cystic fibrosis; central nervous system diseases including Parkinson's disease, Alzheimer's disease, motor neuron disease, Huntington's disease, cerebral malaria and brain injury from pneumococcal meningitis; metabolic diseases including Type 2 diabetes, atherosclerosis, obesity, gout, pseudo-gout, ocular diseases including those of the ocular epithelium, age-related macular degeneration (AMD), corneal infection, uveitis and dry eye, kidney disease including chronic kidney disease, oxalate nephropathy and diabetic nephropathy, liver disease including non-alcoholic steatohepatitis and alcoholic liver disease; inflammatory reactions in skin including contact hypersensitivity and sunburn, inflammatory reactions in the joints including osteoarthritis, juvenile idiopathic arthritis, adult-onset Still's disease, relapsing polychondritis; viral infections including alpha virus (Chikungunya, Ross River) and flavivirus (Dengue and Zika Virus), flu, HIV, hidradenitis suppurativa (HS) and other cyst-causing skin diseases; cancers including lung cancer metastasis, pancreatic cancers, gastric cancers, myelodisplastic syndrome, leukemia, polymyositis, stroke, myocardial infarction, Graft versus Host Disease, hypertension, colitis, helminth infection, bacterial infection, abdominal aortic aneurism, wound healing, depression, psychological stress, pericarditis including Dressler's syndrome, ischaemia reperfusion injury and/or any disease where an individual has been determined to carry a germline or somatic non-silent mutation in NLRP3.

In specific embodiments, the NLRP3-associated disease, disorder or condition is a disease, disorder or condition of the respiratory system, such as chronic obstructive pulmonary disorder (COPD), severe steroid-resistant asthma (SSR), asbestosis, silicosis or cystic fibrosis.

In specific embodiments, the NLRP3-associated disease, disorder or condition is an inflammatory disease, disorder or condition, optionally caused or initiated by a pathogen, such by a viral infection as outlined in further detail below.

In further specific embodiments, the NLRP3-associated disease, disorder or condition is caused by, or is associated with a pathogen. In general, the pathogen may be selected from the group consisting of a virus, a bacterium, a protist, a worm, a fungus and other organisms capable of infecting a mammal.

In further specific embodiments, however, the NLRP3-associated disease, disorder or condition to be treated or prevented according to the present invention is a viral infection or a disease, disorder or condition resulting from a viral infection.

According to these specific embodiments, the medically active liquids for use, methods or uses according to the present invention allows for the treatment or prevention, preferably for the treatment of a viral infection in a patient or subject. Such viral infections may be selected from a broad variety of viral infections including coronavirus, influenza virus, rhinovirus, and adenovirus, such as SARS viruses, MERS viruses, H1N1 influenza, and Avian Flu H5N1, specifically severe acute respiratory syndrome viruses (SARS) such as severe acute respiratory syndrome coronaviruses (SARS-CoV or SARS-CoV-2), Middle East respiratory syndrome viruses such as Middle East respiratory syndrome coronaviruses (MERS-CoV). In specific embodiments, however, the viral infection to be prevented or treated by the method of the present invention is an infection by a coronavirus. In some embodiments, the viral infection is an infection of the respiratory tract, more specifically of the lower respiratory tract such as a pulmonary infection (e.g., a pneumonia).

In further specific embodiments, the viral infection to be treated or prevented according to the present invention is a severe acute respiratory syndrome (SARS), more specifically a SARS-CoV or SARS-CoV-2 virus infection. A SARS-CoV-2 viral infection is believed to be the cause of the pandemic disease COVID-19. Accordingly, in specific embodiments, the medically active liquids for use, methods or uses according to the present invention allow for the treatment of viral infections and/or the diseases, disorders or conditions associated with or caused by such viral infection in a subject or patient diagnosed with COVID-19.

In further specific embodiments as mentioned above, the disease, disorder or condition to be treated or prevented according to the present invention is a lower respiratory tract infection, affecting at least a part of the lower respiratory tract of a subject, specifically a human, such as one or both lungs of a subject or patient (e.g., a pneumonia). According to these embodiments, the NLRP3-associated disease, disorder or condition may be a pulmonary disease, disorder or condition, whereas the term “pulmonary” means that such disease affects or is associated with one or both lungs of a subject or patient.

Specifically, the NLRP3-associated disease, disorder or condition to be treated or prevented according to the present invention is a severe acute respiratory syndrome (SARS), more specifically a SARS-CoV-2 viral infection.

In specific embodiments, as outlined above, the subject to be treated according to the present invention preferably is a human or warm-blooded animal, especially a human. In case of a viral infection or a disease, disorder or condition resulting from such viral infection, in specific embodiments, the subject is diagnosed with a viral infection, such as a coronavirus infection, especially by a SARS or MERS coronavirus. In further specific embodiments, the subject is diagnosed with COVID-19.

In further specific embodiments, the NLRP3-associated disease, disorder or condition to be treated or prevented according to the present invention may be a disease, disorder or condition that results or is caused by an initial infection with a pathogen, especially a viral pathogen. Such NLRP3-associated disease, disorder or condition comprise but are not limited to inflammations or informational processes caused by such an infection such as pneumonia caused by an infection with a coronavirus such as SARS-CoV or SARS-CoV-2.

According to the present invention, the medically active liquid is administered to a subject in nebulized form by inhalation, wherein the medically active liquid comprises a NLRP3 inhibitor and wherein the medically active liquid is administered in nebulized form using an inhalation device.

The medically active liquid to be administered according to the present invention comprises a NLRP3 inhibitor, or in other words, at least one NLRP3 inhibitor, such as a combination of two or more different NLRP3 inhibitors, which can be selected from a broad variety of NLRP3-inhibitors, such as NLRP3-inhibitors disclosed e.g. in WO 2016/131098 A1, WO 2017/184624 A1, or WO 2020/010140 A1, the disclosures of each of which are incorporated herein by reference. In specific embodiments, the chosen NLRP3 inhibitor to be administered to the patient or subject is an inhalable NLRP3 inhibitor.

In specific embodiments, the NLRP3 inhibitor to be administered and comprised by the medically active liquid according to the present invention is a NLRP3 inflammasome inhibitor. In further embodiments, the NLRP3 inhibitor to be administered and comprised by the medically active liquid is a NLRP3 inhibitor which inhibits NLRP3 inflammasome formation. In yet further embodiments, the NLRP3 inhibitor to be administered and comprised by the medically active liquid is a NLRP3 inhibitor which inhibits NLRP3 inflammasome formation activation.

The term “NLRP3 inhibitor” as used herein is to be understood in broad sense und is meant to describe a compound that at least partly inhibits or reduces the activity of the NOD-like receptor family, pyrin domain-containing protein 3 (NLRP3), irrespective of the specific mode of interaction. Accordingly, in specific embodiments, the NLRP3 inhibitor to be administered and comprised by the medically active liquid according to the present invention, preferably in a pharmaceutically effective amount, may be a direct inhibitor of the NLRP3 protein.

Examples of such direct NLRP3 inhibitors comprise but are not limited to MCC950 (N-(1,2,3,5,6,7-hexahydro-s-indacen-4-ylcarbamoyl)-4-(2-hydroxy-2-propanyl)-2-furansulfonamide, sodium; CAS Nr. [256373-96-3]), 3,4-Methylene dioxy-β-nitrostyrene (MNS; CAS Nr. [1485-00-3]), CY-09 (4-[[4-Oxo-2-thioxo-3-[[3-(trifluoromethyl)phenyl]methyl]-5-thiazolidinylidene]methyl] benzoic acid; CAS Nr. [1073612-91-5]), N-[3′,4-dimethoxycinnamoyl]-anthranilic acid (Tranilast; CAS Nr. [53902-12-8]), OLT1177 (3-methylsulfonylpropanenitrile; Dapansutrile; CAS Nr. [54863-37-5]) and Oridonin (7a,20-Epoxy-1a,6b,7,14-tetrahydroxy-Kaur-16-en-15-one, Isodonol, CAS Nr. [28957-04-2]). In the context of the present invention an example of an especially preferred direct NLRP3 inhibitor is MCC950 in the form of its sodium salt as defined above and having the following formula:

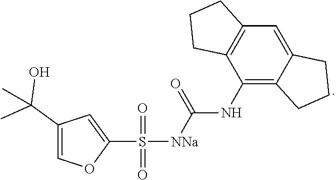

In further embodiments, the NLRP3 inhibitor to be administered and comprised by the medically active liquid according to the present invention may be an indirect NLRP3 inhibitor. Examples of such indirect NLRP3 inhibitors comprise but are not limited to Glyburide (5-chloro-N-(4-[N-(cyclohexylcarbamoyl) sulfamoyl]phenethyl)-2-methoxybenzamide; Gilbenclamide, CAS Nr. [10238-21-8]), 16673-34-0 (4-[2-(5-Chloro-2-methoxybenzamido)ethyl]benzenesulfonamide), JC124 (5-chloro-2-methoxy-N-(4-(N-methylsulfamoyl)phenethyl)benzamide; CAS Nr. [1638611-48-9]) and 1-ethyl-5-methyl-2-phenyl-1H-benzo[d]imidazole (FC11A-2; CAS Nr. [960119-75-9]).

In yet further embodiments, the NLRP3 inhibitor to be administered and comprised by the medically active liquid according to the present invention may be an inhibitor for the constituents of a NLRP3 inflammasome. Examples for such inhibitors for the constituents of a NLRP3 inflammasome comprise but are not limited to Parthenolide (1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one; CAS Nr. [20554-84-1]), VX-740 ((4S,7S)—N-[(2R,3S)-2-ethoxy-5-oxooxolan-3-yl]-7-(isoquinoline-1-carbonylamino)-6,10-dioxo-2,3,4,7,8,9-hexahydro-1H-pyridazino[1,2-a]diazepine-4-carboxamide; Pralnacasan; CAS Nr. [192755-52-5]), VX-765 ((S)-1-((S)-2-(4-amino-3-chlorobenzamido)-3,3-dimethylbutanoyl)-N-((2R,3S)-2-ethoxy-5-oxotetrahydrofuran-3-yl)pyrrolidine-2-carboxamide; Belnacasan; CAS Nr. [273404-37-8]), Bay 11-7082 ((E)-3-(4-Methylphenylsulfonyl)-2-propenenitrile; CAS Nr. [19542-67-7]) and O-hydroxybutyrate (BHB, CAS Nr. [9028-38-0]). As used herein, the constituents of a NLRP3 inflammasome include NLRP3, ASC (apoptosis-associated speck-like protein containing a caspase recruitment domain), and procaspase-1.

As mentioned above, suitable NLRP3 inhibitors comprise e.g., sulfonylureas and other related compounds as disclosed in WO 2016/131098 A1, or WO 2017/184624 A1, or WO 2020/010140 A1 and others, the disclosures of each of which are incorporated herein by reference in their entireties. In specific embodiments, a NLRP3 inhibitor to be administered and comprised by the medically active liquid according to the present invention may be selected from the group consisting of Glyburide, 16673-34-0, JC124, 1-ethyl-5-methyl-2-phenyl-1H-benzo[d]imidazole (FC11A-2), Parthenolide, VX-740, VX-765, Bay 11-7082, (3-hydroxybutyrate (BHB), sulfonylureas such as MCC950, MCC7840 (CAS Nr. [1995067-59-8]), 3,4-Methylenedioxy-p-nitrostyrene (MNS), CY-09, N-[3′,4′-dimethoxycinnamoyl]-anthranilic acid (Tranilast), OLT1177, and Oridonin, specifically selected from the group consisting of MCC950, MCC7840 and Bay 11-7082, especially MCC950.

In some specific embodiments, the NLRP3 inhibitor is a non-polymeric, preferably small molecule, specifically with a molecular weight of from about 100 Da up to about 1200 Da, or from about 150 Da up to about 1000 Da, or up to about 800 Da, or of up to about 750 or 700 Da.

If present as a liquid, the selected NLRP3 inhibitor can be used as such as the medically active liquid to be administered in nebulized form according to the present invention. In alternative embodiments, however, the medically active liquid or, in other words, liquid pharmaceutical composition to be administered according to the present invention and comprising a NLRP3 inhibitor is preferably formulated as a composition that is suitable, and adapted for inhalative use, in other words a composition that may be nebulized or atomized for inhalation and that is physiologically acceptable for inhalation by a subject, specifically by a human.

The medically active liquid or pharmaceutical composition to be administered by inhalation according to the invention may be in the form of a dispersion, for example a suspension with a liquid continuous phase, and a solid dispersed phase or in the form of an emulsion with a liquid continuous phase and a liquid dispersed phase or in the form of a solution. In preferred embodiments, the medically active liquid comprising a NLRP3 inhibitor is provided in the form of a solution. In these cases, the medically active liquid may comprise a solvent or, in other words, a liquid vehicle as the solvent or continuous phase. In many cases, a suitable solvent or liquid vehicle may be an aqueous solvent system comprising water as the only or at least as one of the solvents comprised by the medically active liquid, optionally together with other physiologically acceptable solvents that are suitable for nebulization and inhalative administration to a subject, specifically to a human subject or patient. Further physiologically acceptable solvents that are suitable for nebulization and inhalative administration to a subject comprise but are not limited to alcohols, specifically alcohols with 2 to 4, or preferably 2 or 3, carbon atoms, such as ethanol, propanol or iso-propanol or glycols such as propylene glycol, glycerol, lipophilic liquids such as semi-fluorinated alkanes. The solvents can be used in pure form or in the form of a mixture of two or more of the above-described solvents, optionally together with water as a further co-solvent to form an aqueous solvent system or, in other words, liquid vehicle.

Accordingly, in some embodiments the solvent system or liquid vehicle of the medically active liquid may comprise an alcohol as described above, especially ethanol, propanol, iso-propanol or propylene glycol as the only or dominating solvent. In these cases also, water may be present as a co-solvent, for example in an ethanolic solvent system comprising water, e.g., in an amount of up to about 50 wt.-%, or of up to about 25 wt.-%, or of up to about 10 wt.-% or lower, or in other cases propylene glycol comprising minor amounts of water, such as of up to about 50 wt.-%, or of up to about 25 wt.-%, or of up to about 10 wt.-% or lower. In exemplary embodiments, the medically active liquid may comprise ethanol in an amount of up to about 80 wt.-% or up to about 90 wt.-% and water in an amount of up to about 20 wt.-% or up to about 10 wt.-%, respectively.

In further embodiments, the medically active liquid or liquid pharmaceutical composition may comprise, optionally, one or more physiologically acceptable excipients, which are suitable for inhalative use. Excipients which may be used in the medically active liquid or liquid composition include, but are not limited to, one or more buffering agents to regulate or control pH of the solution, chelating agents, salts such as sodium chloride, taste-masking agents, surfactants, lipids, antioxidants, and co-solvents, which may be used to enhance or improve solubility.

Suitable excipients are known to the skilled person and are described, e.g., in standard pharmacopoeias such as U.S.P. or Ph. Eur., or in the Handbook of Pharmaceutical Excipients, 6th ed. Rowe et al, Eds.; The Pharmaceutical Press and the American Pharmaceutical Association: 2009.

Exemplary compounds suitable as buffers for the adjustment of the pH of the present pharmaceutical compositions comprise, for example, sodium dihydrogen phosphate dihydrate and/or disodium hydrogen phosphate dodecahydrate, sodium hydroxide solution, basic salts of sodium, calcium or magnesium such as, for example, citrates, phosphates, acetates, tartrates, lactates etc., amino acids, acidic salts such as hydrogen phosphates or dihydrogen phosphates, especially those of sodium, moreover, organic and inorganic acids such as, for example, hydrochloric acid, sulphuric acid, phosphoric acid, citric acid, cromoglycinic acid, acetic acid, lactic acid, tartaric acid, succinic acid, fumaric acid, lysine, methionine, acidic hydrogen phosphates of sodium or potassium, etc., and further buffer systems.

In further specific embodiments, the medically active liquid to be nebulized and administered according to the present invention may comprise one or more further excipient which are selected from chelating agents, for example, disodium edetate dihydrate, calcium sodium EDTA, preferably disodium edetate dihydrate.

In yet further specific embodiments, the medically active liquid to be nebulized and administered according to the present invention may comprise one or more preservatives and/or antioxidants. Suitable preservatives comprise but are not limited to benzalkonium chloride (BAC), parabens such as methylparaben, ethylparaben, propylparaben, sodium benzoate, sorbic acid and salts thereof. In specific embodiments, the medically active liquid to be nebulized and administered according to the present invention comprises benzalkonium chloride as a preservative. Suitable antioxidants comprise but are not limited to butylated hydroxytoluene (BHT), vitamin A, vitamin E, vitamin C, retinyl palmitate and others.

Further excipients that may be included in the medically active liquid comprising a NLRP3 inhibitor to be administered according to the present invention comprise, but are not limited to phoshatidylcholines, such as dilauroylphosphatidylcholine (DLPC), dipalmitoylphosphatidylcholine (DPPC), distearoylphosphatidyl glycerol (DTPA), diethylene triamine pentaacetic acid, hydrogenated soy phosphatidylcholine (HSPC) and soy phosphatidylcholine (SPC).

The medically active liquid comprising a NLRP3 inhibitor to be administered to a subject in need thereof by inhalation may, in further embodiments, additionally comprise at least one further medically active compound or active pharmaceutical ingredient (API). Such further medically active compound may be, e.g. be selected from the group consisting of caspase inhibitors, SGK1 Inhibitors and/or further NLRP3 inhibitors as described above, or others.

The amount of the at least one NLRP3 inhibitor comprised by the medically active liquid and to be administered to a patient or subject in need thereof may be determined according to routine experimentation as known to those of skill in the art.

The medically active liquid comprising a NLRP3 inhibitor to be administered according to the method of the present invention may be administered in 1 single or several separate doses by inhalation, such as 1 to about 6 or 4 doses per day, or 2 or 3 doses per day using an inhaler or inhalation device as described in further detail below. In specific embodiments, one dose of the medically active liquid comprises the selected NLRP3 inhibitor, or the selected combination of NLRP3 inhibitors in an amount selected within the range of from about 100 μg to about 2,000 μg (two thousand micrograms), specifically from about 200 μg to about 1,500 μg or even more specifically from about 300 μg to about 1,000 μg, wherein the amount chosen and administered my vary depending on the pharmacological potency as well as on the molecular weight of the chosen NLRP3 inhibitor(s).

In further specific embodiments in which the NLRP3 inhibitor is MCC950, one dose of the medically active liquid to be nebulized and dispensed according to the present invention comprises from about 50 μg to about 500 μg of MCC950, or from about 100 μg to about 400 μg of MCC950.

In further specific embodiments, the medically active liquid comprising a NLRP3 inhibitor preferably, preferably MCC950, MCC7840 or Bay 11-7082, especially MCC950, is dispensed and/or administered in an amount of at least about 1 μL, 2 μL, 5 μL, 10 μL, or 15 μL, or at least about 20 μL, 25 μL, 30 μL, or 50 μL or from about 1 μL to about 50 μL or from about 2 μL to about 30 μL, or from about 5 μL to about 25 μL, or from about 10 μL to about 20 μL. In some embodiments, the medically active liquid comprising the NLRP3 inhibitor, preferably MCC950, MCC7840 or Bay 11-7082, especially MCC950, is dispensed and/or administered in an amount of about 15 μL.

In further specific embodiments, the medically active liquid according to the present invention may comprise the selected NLRP3 inhibitor, preferably MCC950, MCC7840 or Bay 11-7082, especially MCC950, or the selected combination of NLRP3 inhibitors in a concentration selected within the range of from about 5 μg/μL to about 100 μg/μL, such as from about 7.5 μg/μL to about 90 μg/μL, or even from about 10 μg/μL to about 85 μg/μL, especially in cases in which a binary solvent system as described above comprising ethanol and water as the only solvents are used as the liquid vehicle.

In some embodiments in which the selected NLRP3 inhibitor is MCC950 the concentration of MCC 950 in the medically active liquid is selected within the range of from about 50 μg/μL to about 85 μg/μL, or from about 60 μg/μL to about 80 μg/μL, especially in cases in which a binary solvent system as described above comprising ethanol and water as the only solvents are used as the liquid vehicle.

In some embodiments in which the selected NLRP3 inhibitor is Bay 11-7082 the concentration of Bay 11-7082 in the medically active liquid is selected within the range of from about 1 μg/μL to about 15 μg/μL, or from about 2.5 or 5 μg/μL to about 12.5 μg/μL, especially in cases in which a binary solvent system as described above comprising ethanol and water as the only solvents are used as the liquid vehicle.

In specific embodiments, the selected NLRP3 inhibitor or, more specifically, the medically active liquid comprising the selected NLRP3 inhibitor and optionally the further pharmaceutically active components or excipients as described above may be administered for prolonged periods of time such as for several weeks or even months, depending on severity and success of the treatment of the subject in need thereof. In further specific embodiments, however, the NLRP3 inhibitor of the medically active liquid comprising such NLRP3 inhibitor is preferably administered for a period of at least 5 days, such as from 5 to about 14 days or to about 10 days.

According to the compositions for use, methods or uses of the present invention, the medically active liquid comprising a NLRB3-inhibitor is administered to a subject in need thereof in nebulized form using an inhalation device. The term “in nebulized form” as used herein means, with regard to the medically active liquid to be administered, that the medically active liquid is present in the form of an aerosol in which the medically active liquid comprising the NLRP3 inhibitor is present in the form of finely divided particles or droplets dispersed in air or another propellant as the continuous phase.

In specific embodiments, such an aerosol has respirable particles or droplets, preferably having a median diameter, specifically a mass median aerodynamic diameter (as measured by laser diffraction), of not more than about 10 μm, in particular not more than about 7 μm, or not more than about 5 μm, respectively. In some embodiments, the average particle size distribution of the nebulised medically active liquid comprising an NLRP3 inhibitor is about 1.0 μm to about 3.0 μm at the Dv10. In other embodiments, the average particle size distribution of the nebulised medically active aerosol comprising an NLRP3 inhibitor is about 3.0 μm to about 5.0 μm at the Dv50. In yet other embodiments, the average particle size distribution of the nebulised medically active aerosol comprising an NLRP3 inhibitor is about 15 μm to about 25 μm at the Dv90. The terms “Dv10, Dv50, and Dv90” refer to the maximum particle diameter in micrometers (μm) where 10%, 50%, and 90%, respectively, of which the sample volume exists.

In further specific embodiments, the NLRP3 inhibitor comprised by the medically active liquid is administered to the lungs of the subject, specifically in form of a respirable aerosol comprising the NLRP3 inhibitor.

In further specific embodiments, the medically active liquid or composition to be administered according to the present invention may be essentially free of a propellant, such as a hydrofluoroalkane (HFA) propellant.

According to the medically active liquid for use, methods or uses of the present invention, the medically active liquid comprising a NLRP3 inhibitor his administered to a subject in need thereof by inhalation using an inhalation device. The term “inhalation device” as used herein is to be understood in the broadest sense as referring to a device which is configured and adapted for the generation of an inhalable mist, vapor, or spray, or more specifically, allows and is adapted for the nebulization in inhalative administration, preferably by oral inhalation, of a medically active liquid. Examples of such inhalation devices are known to those of skill in the art and comprise, but are not limited to, e.g., metered dose inhalers (MDI), nebulizers, vibrating mesh inhalers and soft-mist-inhalers (SMI). Exemplary embodiments of suitable inhalers for the administration of the medically active liquid comprising a NLRP3 inhibitor are described, e.g. in “Inhalation drug delivery devices: technology update” Medical Devices: Evidence and Research 2015:8 131-139; or “Recent advances in in aerosolized drug delivery”, A. Chandel et al., Biomedicine & Pharmacotherapy, Vol. 112, April 2019, 108601 (https://doi.org/j.biopha.2019.108601), or in “Pharmaceutical Inhalation Aerosol Technology”, Third Edition, A. J. Hickey et al., May 1, 2019, the contents of each of which are herein incorporated by reference in their entireties.

In specific embodiments, such nebulization and administration by inhalation of the medically active liquid comprising a NLRP3 inhibitor can be performed using a hand-held inhalation device.

In further specific embodiments, the inhalation device that may be used to administer the medically active liquid comprising the NLRP3 inhibitor is a soft-mist-inhaler. The term “soft-mist-inhaler” as used herein, in specific embodiments, refers to a non-electrified mobile inhalation device for liquid formulations with low velocity nebulization properties that allows to generate in inhalable aerosol with droplet sizes or droplet size distributions that allow for a deep penetration of the (droplets of) the medically active liquid comprising a NLRP3 inhibitor into the lungs of the patient or subject. In further specific embodiments, such inhalation device or, more specifically, such soft-mist inhaler comprises at least one impingement-type nozzle as described in further detail below for the nebulization/aerosolization of the medically active liquid comprising the NLRP3 inhibitor.

Suitable inhalation devices are known such as, e.g., the Respimat® inhaler (Boehringer Ingelheim), vibrating membrane nebulizers such as eFlow® (PARI), Vibrating-Mesh® nebulizers (such as Philips InnoSpire Go) and others.

A further exemplary suitable inhalation device is known e.g., from document EP 0 627 230 B1, the contents of which are incorporated herein by reference in its entirety. Essential components of this exemplary inhalation device are a reservoir in which the medically active liquid that is to be aerosolized is contained; a pumping device for generation of a pressure being sufficiently high for nebulizing; as well as an atomizing device in the form of a nozzle. By means of the pumping device, the liquid is drawn in a discrete amount, i.e., not continuously, from the reservoir, and fed to the nozzle. The pumping device works without propellant and generates pressure mechanically. Accordingly, in specific embodiments a preferred inhalation device to be used in the context of the present invention works without a propellant. In further specific embodiments, the pressure of the medically active liquid to be dispensed is generated mechanically, such as by the force of a spring.

A further exemplary embodiment of a suitable inhalation device is described in document WO 91/14468 A1, the contents of which are herein incorporated by reference in its entirety. In such a device, the pressure in the pumping chamber which is connected to the housing is generated by movement of a moveable hollow piston. The piston is moveably arranged inside the immobile cylinder or pumping chamber. The (upstream arranged) inlet of the hollow piston is fluidically connected to the interior of the reservoir (reservoir pipe section). Its (downstream arranged) tip leads into the pumping chamber. Furthermore, a check valve that inhibits a back flow of liquid into the reservoir is arranged inside the tip of the piston.

Soft-mist inhalers as described above have been proven as a very effective means for providing medically active liquids or compositions or pharmaceutically active compounds contained therein into the lungs of a patient or subject in need thereof. Such a soft mist inhaler usually comprises one or a plurality of impingement-type nozzles. Such an impingement-type nozzle is adapted to emit at least two jets of liquid which are directed such as to collide and break up into small aerosol droplets of the medically active liquid to nebulized. Accordingly, the term “impingement-type nozzle” as used herein refers to a nozzle having at least two liquid channels adapted and arranged to emit at least two jets of the liquid to be nebulized or aerosolized, wherein the at least two liquid jets are directed such as to collide and to break up into droplets of the medically active liquid as described above. The nozzle or nozzles usually are firmly affixed to the user-facing side of the housing of the inhalation device in such a way that it is immobile, or non-moveable, relative to the housing or at least relative to the side or part of the housing which faces the user (e.g., patient) when the device is used.

A specific embodiment of such a soft mist inhaler which is suitable for the administration of the medically active liquid comprising a NLRP3 inhibitor is described, e.g., in international patent application WO 2018/197730 A1, the contents of which are incorporated herein by reference in its entirety. It should be noted, however, that the inhaler device described therein is just one example of a suitable inhaler device to be used according to the present invention and, therefore, should not be interpreted as limiting the scope of the invention in any respect.

In specific embodiments, the inhalation device that may be used in the context of the present invention to administer the medically active liquid comprising a NLRP3 inhibitor may be in inhalation device, specifically a hand-held inhalation device for delivering a nebulised medically active aerosol for inhalation therapy, comprising

(a) a housing having a user-facing side;

(b) an impingement-type nozzle for generating the nebulised aerosol by collision of at least two liquid jets, the nozzle being firmly affixed to the user-facing side of the housing such as to be immobile relative to the housing;

(c) a fluid reservoir arranged within the housing; and

(d) a pumping unit arranged within the housing, the pumping unit having
an upstream end that is fluidically connected to the fluid reservoir;
a downstream end that is fluidically connected to the nozzle;
wherein the pumping unit is adapted for pumping fluid from the fluid reservoir to the nozzle;

wherein the pumping unit further comprises

(i) a riser pipe having an upstream end, wherein the riser pipe is
adapted to function as a piston in the pumping unit, and
firmly affixed to the user-facing side of the housing such as to be immobile relative to the housing; and

(ii) a hollow cylinder located upstream of the riser pipe, wherein the upstream end of the riser pipe is inserted in the cylinder such that the cylinder is longitudinally movable on the riser pipe; and

(iii) a lockable means for storing potential energy when locked and for releasing the stored energy when unlocked, the means being arranged outside of, and mechanically coupled to, the cylinder such that unlocking the means results in a propulsive longitudinal movement of the cylinder towards the downstream end of the pumping unit.

In specific embodiments, such a preferred inhalation device comprises a housing having a user-facing side, an impingement-type nozzle for generating the nebulised aerosol by collision of at least two liquid jets, a fluid reservoir arranged within the housing, and a pumping unit which is also arranged within the housing. The nozzle may be firmly affixed to the user-facing side of the housing such as to be immobile relative to the housing. In these preferred embodiments, the pumping unit may have an upstream end that is fluidically connected to the fluid reservoir and a downstream end that is fluidically connected to the nozzle, whereas in the context of the present invention an “upstream” direction or position means a position or direction from which the medically active liquid is conveyed, and a “downstream” direction or position means a position or direction to which the medically active liquid is conveyed or in other words in the direction of the nozzle. Furthermore, the pumping unit may be adapted for pumping fluid from the fluid reservoir to the nozzle, and it may comprise a riser pipe which is adapted to function as a piston in the pumping unit, a hollow cylinder and a lockable means for storing potential energy. The riser pipe is preferably firmly affixed to the user-facing side of the housing such as to be immobile relative to the housing. The hollow cylinder may be located upstream of the riser pipe, and the upstream end of the riser pipe may be inserted in the cylinder such that the cylinder is longitudinally movable on the riser pipe. The lockable means typically is capable of storing potential energy when locked and is adapted for releasing the stored energy when unlocked. The lockable means may be arranged outside of, and mechanically coupled to, the cylinder in such a way that unlocking the means results in a propulsive longitudinal movement of the cylinder towards the downstream end of the pumping unit.

As used herein, a “hand-held” inhalation device is a mobile inhalation device which can be conveniently held in one hand (preferably by the user but also by another person) and which is suitable for delivering a nebulised medically active aerosol for inhalation therapy. In order to be suitable for inhalation therapy, the device must be able to emit a medically active aerosol whose particle size is respirable, i.e., small enough to be taken up by the lungs of a patient or user, as already outlined above. Typically, respirable particles have a diameter, specifically a diameter as measured by laser diffraction of not more than about 10 μm, in particular not more than about 7 μm, or not more than about 5 μm, respectively with particle size distributions as described in detail above. In this respect, inhalation devices suitable the administration of the medically active liquid in nebulized form according to the present invention are also substantially different from devices that emit a spray for oral or nasal administration, such as disclosed in US 2004/0068222 A1, the contents of which are herein incorporated by reference in its entirety.

The inhalation device that may be used according to the present invention is capable of delivering a nebulised aerosol or, more specifically, the medically active liquid comprising an NLRP3 inhibitor in nebulized form. As used herein, an aerosol is a system having at least two phases: a continuous phase which is gaseous, and which comprises a dispersed liquid phase in the form of small liquid droplets. Optionally, the liquid phase may itself represent a liquid solution, dispersion, suspension, or emulsion. In specific embodiments, the gaseous phase of the medically active liquid in aerosolized form according to the present invention is air or another physiologically acceptable gas or a mixture thereof, preferably air.

A suitable nozzle is important for the generation of a nebulised aerosol. According to specific embodiments of the invention, the nozzle of preferred inhalation devices, specifically soft-mist inhalers as described above preferably is of the impingement type. This means that the nozzle is adapted to emit at least two jets of medically active liquid which are directed such as to collide and break up into small aerosol droplets. The nozzle may be firmly affixed to the user-facing side of the housing of the inhalation device in such a way that it is immobile, or non-moveable, relative to the housing or at least relative to the side or part of the housing which faces the user (e.g., patient) when the device is used.

The fluid reservoir of the specific hand-held inhalation device as described above which may be arranged within the housing may be adapted to hold or store the medically active liquid comprising an NLRP3 inhibitor from which the nebulised aerosol is generated and delivered by the inhalation device.

The pumping unit of the specific inhalation device which may also be arranged within the housing may be preferably adapted to function as a piston pump, also referred to as plunger pump, wherein the riser pipe may function as the piston, or plunger, which is longitudinally moveable within the hollow cylinder. In this embodiment, the inner segment of the hollow cylinder in which the upstream end of the riser pipe moves may form a pumping chamber which has a variable volume, depending on the position of the riser pipe relative to the cylinder.

The hollow cylinder of the preferred inhalation device which provides the pumping chamber may be fluidically connected with the fluid reservoir, either directly or indirectly, such as by means of an optional reservoir pipe (or reservoir pipe section). Similarly, the riser pipe, whose reservoir-facing, interior (upstream) end which can be received in the hollow cylinder, may be fluidically connected at its downstream or exterior end to the nozzle in a liquid-tight manner, either directly or indirectly.

In this context, the expression “hollow cylinder” as used herein refers to a part or member which is hollow in the sense that it comprises an internal void which has a cylindrical shape, or which has a segment having a cylindrical space. In other words, and as is applicable to other types of piston pumps, it is not required that the external shape of the respective part or member is cylindrical. Moreover, the expression “hollow cylinder” does not exclude an operational state of the respective part or member in which the “hollow” space may be filled with material, e.g., with a liquid to be nebulised.

As used herein, a “longitudinal movement” is a movement along the main axis of the hollow cylinder, and a propulsive movement is a movement of a part in a downstream (or forward) direction.

In some embodiments, the riser pipe of the pumping unit of the preferred hand-held inhalation device is arranged downstream of the cylinder, and it is preferably firmly affixed to the user-facing side of the housing such as to be immobile relative to the housing or at least to the part of the housing which comprises the user-facing side of the housing. For the avoidance of doubt, the term “firmly fixed” as used in this context herein means either directly or indirectly (i.e., via one or more connecting parts) fixed such as to prevent relative movement between the respective parts. As in the preferred inhalation device as described above the nozzle is also immobile relative to the housing or the respective part of the housing, the riser pipe is also immobile relative to the nozzle, and the pumping action is affected by the longitudinal movement of the hollow cylinder. A propulsive movement of the cylinder, which is arranged in an upstream position relative to the riser pipe, results in a decrease of the volume of the pumping chamber, and a repulsive movement of the cylinder results in an increase of the volume. In other words, in the preferred hand-held inhalation device the riser pipe maintains its position relative to the housing, and the hollow cylinder can alter its position relative to the housing, and in particular, along a longitudinal axis of the same, such as to perform a piston-in-cylinder-type movement of the immobile riser pipe in the moveable cylindrical member.

This arrangement differs from other impingement-type inhalation devices which rely on a pumping unit whose riser pipe is in an upstream position and a cylindrical member in a downstream position wherein the riser pipe is moveable and the cylindrical member is fixed to the housing, as disclosed in US 2012/0090603 A1, the contents of which are incorporated herein by reference in its entirety. It should be noted, however, that inhalation devices with this type of pumping may also be suitable for the nebulization an inhalative administration of the medically active liquid comprising an NLRP3 inhibitor according to the present invention.

A key advantage of the described preferred inhalation device is that the passage between pumping chamber and fluid reservoir can be designed with less restrictions with respect to its dimensions. It is, for example, possible to accommodate a significantly larger inlet valve (also referred to as check valve), which is easier to manufacture since it does not have to be contained within a narrow riser pipe. Instead, the arrangement allows the use of a check valve whose size is only restricted by the interior size of the housing or the dimensions of the means for storing potential energy. In other words, the diameters of the valve, the riser pipe and—if used—the reservoir pipe do not need to match each other. Furthermore, since no movable piston needs to be connected to the fluid reservoir, the component which provides the fluid connection to the reservoir can be designed independently of the moveable component, i.e., the hollow cylinder, allowing the individual parts to be adapted to suit their respective individual functions. In this respect, the described pump arrangement provides for higher design flexibility because the moveable hollow cylinder, due to its robust structure and dimensions, provides better opportunities for designing a mechanically stable connection with the reservoir than would a less robust moveable riser pipe. Also, the connection between the hollow cylinder and the fluid reservoir can be designed with a larger diameter, such that higher flow velocities and fluid viscosities become feasible. Further, a support for the reservoir can be integrated into any component that comprises the cylinder. Additionally, any vent for pressure equilibration of the reservoir can be moved away from the reservoir body itself to a connector which forms an interface between reservoir and hollow cylinder, thus facilitating construction and avoiding the necessity to provide an essentially “open” reservoir body.

As already mentioned, the lockable means for storing potential energy of the described preferred inhalation device may be adapted to store energy in its locked state and to release the stored energy when unlocked. In specific embodiments, the lockable means may be mechanically coupled to the hollow cylinder in such a way such that unlocking the means results in a propulsive longitudinal movement of the cylinder towards the downstream end of the pumping unit. During this movement, the internal volume of the cylinder, i.e., the volume of the pumping chamber, decreases. Vice versa, when the means for storing potential energy is in the locked state, the hollow cylinder is in its most upstream position in which the volume of the pumping chamber is largest. The locked state could also be considered a primed state. When the state of the means for storing energy is altered from the unlocked to the locked state, which could be referred to as priming the device, the hollow cylinder performs a repulsive longitudinal movement, i.e., from its most downstream position towards its most upstream position. A pumping cycle of the preferred inhalation device as described above usually consists of two subsequent and opposing movements of the cylinder starting from its most downstream position to its most upstream (or primed) position and—driven by the means for storing potential energy that now releases its energy—back to its most downstream position.

In specific embodiments, the inhalation device suitable for the generation of the medically active liquid in nebulized form according to the present invention is capable, especially in the case of inhalation device having an impingement-type nozzle is capable of pressurizing the medically active liquid to be nebulized to a pressure of up to 1,000 bar (one thousand bar), such as from about 2 bar to about 500 bar or to about 300 bar or from about 50 bar to about 250 bar.

In specific embodiments of the preferred inhalation device as described above, the pumping unit is a high-pressure pumping unit and adapted to operate, or to expel fluid, at a pressure of at least about 50 bar. In other preferred embodiments, the operating pressure of the pumping unit is at least about 10 bar, or at least about 100 bar, or from about 2 bar to about 1,000 bar, or from about 50 bar to about 250 bar, respectively. As used herein, the “operating pressure” is the pressure at which the pumping unit expels fluid, in particular the medically active liquid comprising a NLRP3 inhibitor, such as an inhalable aqueous liquid formulation of a NLRP3 inhibitor as described above, from its pumping chamber in a downstream direction, i.e., towards the nozzle. In this context, the expression “adapted to operate” means that the components of the pumping unit are selected with respect to the materials, the dimensions, the quality of the surfaces and the finish are selected such as to enable operation at the specified pressure.

Moreover, such high-pressure pumping unit implies that the means for storing potential energy is preferably capable of storing and releasing a sufficient amount of energy to drive the propulsive longitudinal movement of the cylinder with such a force that the respective pressure is obtained.

For example, in the preferred inhalation device as described herein the means for the storage of potential energy may be designed as a tension or pressure spring. Alternatively, besides a metallic or plastic body, also a gaseous medium, or magnetic force utilizing material can be used as means for energy storage. By compressing or tensioning, potential energy may be fed to the means. One end of the means may be supported at or in the housing at a suitable location; thus, this end is essentially immobile. With the other end, it may be connected to the hollow cylinder which provides the pumping chamber; thus, this end is essentially moveable. The means can be locked after being loaded with a sufficient amount of energy, such that the energy can be stored until unlocking takes place. When unlocked, the means can release the potential energy (e.g., spring energy) to the cylinder with the pumping chamber, which is then driven such as to perform a (in this case, longitudinal) movement. Typically, the energy release takes place abruptly, so that a high pressure can build up inside the pumping chamber before a significant amount of liquid is emitted, which results in a pressure decrease. In the preferred inhalation device as described above, during a significant portion of the ejection phase, an equilibrium exists of pressure delivered by the means for the storage of potential energy, and the amount of already emitted liquid. Thus, the amount of liquid remains essentially constant during this phase, which is a significant advantage to devices which use manual force of the user for the emission, such as the devices disclosed in documents US 2005/0039738 A1, US 2009/0216183 A1, US 2004/0068222 A1, or US 2012/0298694 A1, the contents of each of which are incorporated by reference in their entireties, since manual force depends on the individual user or patient and is very likely to vary largely during the ejection phase, resulting in inhomogeneous droplet formation, size, and amount. In contrast to these devices, the means according to the preferred inhalation device as described above in connection with the present invention ensures that the inhalation device delivers highly reproducible results.

The means for storing potential energy may also be provided in the form of a highly pressurized gas container. By suitable arrangement and repeatable intermittent activating (opening) of the same, part of the energy which is stored inside the gas container can be released to the cylinder. This process can be repeated until the remaining energy is insufficient for once again building up a desired pressure in the pumping chamber. After this, the gas container must be refilled or exchanged.

In one of the preferred embodiments, the means for storing potential energy comprised by the inhalation device that may be used in the context of the present invention is a spring having a load of at least 10 N in a deflected state. In a particularly preferred embodiment, the means for storing potential energy is a compression spring made of steel having a load from about 1 N to about 500 N in its deflected state. In other preferred embodiments, the compression spring from steel has a load from about 2 N to about 200 N, or from about 10 N to about 100 N, in its deflected state.

The inhalation device that may be used in connection with the method of the present invention is preferably adapted to deliver the nebulised medically active aerosol (i.e., the medically active liquid comprising a NLRP3 inhibitor in nebulized form) in a discontinuous manner, i.e., in the form of discrete units, wherein one unit is delivered per pumping cycle. In this aspect, suitable inhalation devices differ from commonly known nebulisers such as jet nebulisers, ultrasonic nebulisers, vibrating mesh nebulisers, or electrohydrodynamic nebulisers which typically generate and deliver a nebulised aerosol continuously over a period of several seconds up to several minutes, such that the aerosol requires a number of consecutive breathing manoeuvres in order to be inhaled by the patient or user. Instead, a preferred inhalation device suitable for the administration of the medically active liquid according to the present invention is preferably adapted to generate and emit discrete units of aerosol, wherein each of the units corresponds to the amount (i.e., volume) of fluid (i.e., medically active liquid) which is pumped by the pumping unit in one pumping cycle into the nozzle where it is immediately aerosolised and delivered to the user or patient. Vice versa, the amount of medically active liquid pumped by the pumping unit in one pumping cycle determines the amount of the pharmacologically active agent which the patient receives per dosing. It is therefore highly important with respect to achieving the desired therapeutic effect that the pumping unit operates precisely, reliably and reproducibly. The inventors have found that especially the preferred inhalation device as described above incorporating the pumping unit as described above is particularly advantageous in that it does exhibit high precision and reproducibility.

In one preferred embodiment, a single dose of the medication (i.e., of the nebulised aerosol of the medically active liquid comprising a NLRP3 inhibitor) is contained in one unit, i.e., in the volume that is delivered from the pumping unit to the nozzle for aerosol generation in one single pumping cycle. In this case, the user or patient will prime and actuate the inhalation device only once, and inhale the released aerosol in one breathing manoeuvre, per dosing (i.e., per dosing event).

In another preferred embodiment, a single dose of the medication consists of two units of the aerosol, and thus requires two pumping cycles. Typically, the user or patient will prime the device, actuate it such as to release and inhale a unit of the aerosol, and then repeat the procedure. Alternatively, three or more aerosol units may constitute a single dosing.

The volume of medically active liquid comprising a NLRP3 inhibitor that is pumped by the pumping unit in one pumping cycle is preferably in the range from about 2 to about 150 μL. In particular, the volume may range from about 0.1 to about 1,000 μL, or from about 1 to about 250 μL, or from about 1 to about 100 μL, or from about 2 to about 50 μL, or from about 5 to about 25 μL, respectively. These volume ranges are nearly the same as the volume of liquid phase that is contained in one unit of aerosol generated by the inhalation device, perhaps with minor differences due to minute losses of liquid in the device.

In another preferred embodiment of the preferred inhalation device as described above, the pumping unit of the inhalation device comprises an inlet valve, also referred to as a check valve or inlet check valve, positioned in the hollow cylinder. According to this embodiment, the interior space of the hollow cylinder, i.e., the pumping chamber, is fluidically connected with the fluid reservoir via the inlet check valve. The inlet valve allows the inflow of liquid into the pumping chamber, but prevents the backflow of liquid towards, or into, the fluid reservoir. The position of the inlet valve may be at or near the upstream end of the cylinder such as to make nearly the entire internal volume of the hollow cylinder available for functioning as the pumping chamber. Alternatively, it may be more centrally located along the (longitudinal) main axis of the hollow cylinder such as to define an upstream segment and a downstream segment of the cylinder, the upstream segment being upstream of the inlet valve and the downstream segment being downstream of the valve. In this case the pumping chamber is located in the downstream segment.

As mentioned, one of the advantageous effects is that an inlet valve having relatively large dimensions may be accommodated in this position, i.e., at the upstream end of the pumping chamber. This is particularly beneficial as it allows for large dimensions of the fluid conduit(s) within the valve, thus enabling high fluid velocities which translate into a rapid filling of the pumping chamber during the priming of the inhalation device. Moreover, the use of liquids having a higher viscosity than ordinary liquid formulations for inhalation, such as highly concentrated solutions of soluble active ingredients, become feasible for inhalation therapy.

According to a further preferred embodiment, the inlet valve may be adapted to open only when the pressure difference between the upstream and the downstream side of the valve, i.e., the fluid reservoir side and the pumping chamber side, is above a predefined threshold value, and remains closed as long as the pressure difference is below the threshold value. The term “pressure difference” as used in this context means that, irrespective of the absolute pressure values, only the relative pressure difference between the two sides is relevant for determining whether the valve blocks or opens. If, for example, the pressure on the upstream (reservoir) side is already positive (e.g., 1.01 bar due to thermal expansion), but the pressure on the downstream (pumping chamber) side is ambient pressure (1.0 bar, no activation of the device), the pressure difference (here: 0.01 bar) is below the threshold value (e.g., 20 mbar), which allows the valve to stay closed even when subject to a positive pressure in opening direction. This means that the check valve remains closed until the threshold pressure is met, thus keeping the passage between reservoir and pumping chamber safely shut e.g., when the inhalation device is not in use. Examples for threshold pressure differences are in the range of 1 to 1,000 mbar, and more preferably between about 10 and about 500 mbar, or between about 1 and about 20 mbar.

When actuating the preferred inhalation device as described above, as the means for storing potential energy alters its state from a locked state to an unlocked state, energy may be released which effects the cylinder to perform its propulsive longitudinal movement, significant pressure is built up in the pumping chamber. This generates a marked pressure difference (due to a high pressure in the pumping chamber and a substantially lower pressure in the fluid reservoir) which exceeds the threshold value of the pressure difference, so that the check valve opens and allows the pressure chamber to become filled with liquid from the reservoir.

A valve type that may be designed to operate with such a threshold pressure difference is, e.g., a ball valve pre-loaded with a spring. The spring pushes the ball into its seat, and only if the pressure acting against the spring force exceeds the latter, the ball valve opens. Other valve types which—depending on their construction—may operate with such a threshold pressure difference are duckbill valves or flap valves.

The advantage of such a valve operating with a threshold pressure difference is that the reservoir can be kept closed until active use is being made of the inhalation device, thus reducing unwanted splashing of reservoir liquid during device transport, or evaporation during long-term storage of the device.

In a further preferred embodiment, the inhalation device that may be used in the context of the invention further comprises an outlet valve inside the riser pipe, or at an end of the riser pipe, for avoiding a return flow of liquid or air from the riser pipe into the hollow cylinder. In many cases, the use of such outlet valve will prove to be advantageous. Typically, the downstream end of the riser pipe is located close to the nozzle. The nozzle is in fluidic communication with the outside air. After emitting in aerosolised form, the amount of liquid which is delivered from the pumping unit through the nozzle, driven by the propulsive longitudinal movement of the cylinder, the pumping chamber must be refilled. For this purpose, it slides back on the riser pipe into its previous upstream position (i.e., performs a repulsive longitudinal movement), so that the interior volume of the pumping chamber increases. Along with this, a negative pressure (sometimes also referred to as “under-pressure”) is generated inside the pumping chamber which causes liquid to be sucked into the pumping chamber from the fluid reservoir which is located upstream of the pumping chamber. However, such negative pressure may also propagate downstream through the riser pipe up to the outside of the nozzle and could lead to air being sucked into the device through the nozzle, or nozzle openings, respectively. This problem can be avoided by providing an outlet valve, also referred to as outlet check valve, which opens towards the nozzle openings and blocks in the opposite direction.

Optionally, the outlet valve is of a type that blocks below (and opens above) a threshold pressure difference as described in the context of the inlet valve above. If a ball valve with a spring is used, the spring force must be directed against the pumping chamber such that when the difference between the interior pressure of the pumping chamber and the ambient pressure exceeds the threshold pressure difference value, the outlet valve opens. The advantages of such a valve correspond to the respective aforementioned advantages.

As mentioned, the outlet valve may be positioned within the riser pipe. Alternatively, the inhalation device may comprise an outlet valve which is not integrated within the riser pipe, but positioned at or near one of the ends of the riser pipe, in particular at or near its downstream end, e.g., in a separate connector between the riser pipe and the nozzle. This embodiment may be advantageous in certain cases, e.g., if there is a need for a riser pipe with a particularly small diameter which makes the integration of a valve difficult. By accommodating the outlet valve downstream of the riser pipe, a valve with a relatively large diameter may be used, thus simplifying the requirements for the valve design.

In a further alternative embodiment, the outlet valve is absent. This embodiment may be feasible as the fluid channels of an impingement-type nozzle may have relatively small cross sections, resulting in only minor or very slow back flow at the given pressure conditions during the priming of the device. If the amount of backflow is considered acceptable in view of a particular product application, the inhaler design may be simplified by avoiding the outlet valve.

In any case, whether the inhalation device is designed with or without an outlet valve, all other options and preferences described with respect to other device features are applicable to both of these alternative embodiments.

In a further preferred embodiment, the inhalation device that may be used in the context of the present invention comprises a fluid reservoir which is firmly attached to the hollow cylinder such as to be moveable together with the hollow cylinder inside the housing. This means that in each ejection phase of the pumping cycle, the fluid reservoir moves together with the hollow cylinder from an initial (“upstream”) position, in which the pumping chamber has its maximum interior volume, towards an end (“downstream”) position, in which the volume of the pumping chamber is minimal; and during the subsequent “priming” step, the fluid reservoir returns together with the hollow cylinder to their initial (“upstream”) position.

As used herein, the expression “firmly attached” includes both permanent and non-permanent (i.e., releasable) forms of attachment. Moreover, it includes direct and indirect (i.e., via one or more connecting parts) types of attachment. At the same time, as mentioned above, “firmly attached” means that the respective parts are fixed to each other in such a way as to substantially prevent their movement relative to each other. In other words, two parts that are firmly attached to each other may only be movable together, and with respect to each other, they are non-movable or immobile.

One of the advantages of this embodiment wherein the fluid reservoir is firmly attached to the hollow cylinder is that it provides the smallest possible dead volume between the reservoir and the pumping chamber.

According to an alternative embodiment, the fluid reservoir may be fluidically connected to the hollow cylinder by means of a flexible tubular element, and firmly attached to the housing. According to this embodiment, the reservoir is not firmly attached to the hollow cylinder and does not move along with it when the cylinder performs its longitudinal movements. Instead, it is firmly, but optionally detachably, directly or indirectly, attached to the housing or to a part of the housing. One advantage of this embodiment is that the energy which is abruptly released upon unlocking the means for storing potential energy solely acts on the hollow cylinder and not on the fluid reservoir. This may be particularly advantageous in cases in which the fluid reservoir in its initial (fully filled state) at the beginning of its usage has a relatively large mass which decreases overuse. A higher acceleration of the hollow cylinder would translate into a higher pressure in the pumping chamber.

For the avoidance of doubt, all other options and preferences described herein-above and below with respect to other device features are applicable to both of these alternatives, i.e., regardless of whether the fluid reservoir is firmly attached to the hollow cylinder or not.

In one embodiment, the fluid reservoir may be designed to be collapsible, such as by means of a flexible or elastic wall. The effect of such design is that upon repeated use of the device which involves progressive emptying of the reservoir, the flexible or elastic wall buckles or folds such as to reduce the internal volume of the reservoir, so that the negative pressure which is necessary for extraction of a certain amount of liquid is not required to increase substantially over the period of use. In particular, the reservoir may be designed as a collapsible bag. The advantage of a collapsible bag is that the pressure inside the reservoir is almost independent of the filling level, and the influence of thermal expansion is almost negligible. Also, the construction of such a reservoir type is rather simple and already well established.

A similar effect can be achieved with a rigid container which has a moveable bottom (or wall) by means of which the interior volume of the reservoir can also be successively reduced.

Soft-mist inhalers such the specific soft-mist inhaler as described in detail above allow for the administration of discrete doses of the medically active liquid comprising a NLRP3 inhibitor in short periods of time as the generation of the aerosol of the medically active liquid to be administered by inhalation is usually completed within a period (also referred to herein as “spray duration” or “event duration”) of up to 3 sec, typically within a period selected within the range of from about 0.5 to about 3 sec, or from about 0.5 or from about 1 to about 2 sec.

In a second aspect, the present invention provides for a method for the treatment or prevention of a NLRP3-associated disease, disorder or condition in a subject, the method comprising the step of administering to said subject a medically active liquid in nebulized form by inhalation, wherein the medically active liquid comprises a NLRP3 inhibitor and wherein the medically active liquid is administered in nebulized form using an inhalation device.

In a third aspect, the present invention provides for the use of NLRP3 inhibitor for the preparation of a medically active liquid for the treatment of a NLRP3-associated disease, disorder or condition, wherein the medically active liquid is administered to a subject in nebulized form by inhalation using an inhalation device.

In a fourth aspect, the present invention provides for the use of a medically active liquid comprising a NLRP3 inhibitor for the prevention or treatment of a NLRP3-associated disease, disorder or condition, wherein the medically active liquid is used by inhalation of the medically active liquid in nebulized form, wherein the medically active liquid in nebulized form is generated by nebulization using an inhalation device.

In a fifth aspect, the present invention provides for the use of an inhalation device for the prevention or treatment of a NLRP3-associated disease, disorder or condition in a subject, wherein the medically active liquid is administered in nebulized form using the inhalation device and wherein the medically active liquid comprises a NLRP3 inhibitor.

In a sixth aspect, the present invention provides for a kit, specifically for a kit for the treatment or prevention of a NLRP3-associated disease, disorder or condition in a subject, the kit comprising
a medically active liquid comprising a NLRP3 inhibitor for the prevention or treatment of a NLRP3-associated disease, disorder or condition, wherein the medically active liquid is adapted to be administered to the subject in nebulized form by inhalation; and
an inhalation device, preferably a hand-held inhalation device, such as a soft-mist-inhaler.

According to this aspect of the invention also, the medically active liquid comprising an NLRP3 inhibitor can be provided in the form of a reservoir as described above containing the medically active liquid.

In a seventh aspect, the present invention provides for the use of a medically active liquid comprising a NLRP3 inhibitor in the manufacture of a kit for the treatment of a NLRP3-associated disease, disorder or condition in a subject, the kit comprising
a medically active liquid comprising a NLRP3 inhibitor for the prevention or treatment of a NLRP3-associated disease, disorder or condition, wherein the medically active liquid is adapted to be administered to the subject in nebulized form by inhalation; and
an inhalation device, preferably a hand-held inhalation device, such as a soft-mist-inhaler.

It should be noted that all embodiments, features and combinations thereof disclosed above in connection with the first aspect of the invention apply equally to all further aspects of the invention.

### DETAILED DESCRIPTION OF THE DRAWINGS

In FIG. 1, one of the preferred embodiments of an inhalation device useful for the method according to the present invention is depicted schematically and not-to-scale. FIG. 1 shows the situation prior to first use.

The inhalation device comprises a housing (1), which is preferably shaped and dimensioned such that it can be held with one hand and can be operated by one finger, e.g., a thumb or index finger (not shown). A fluid reservoir (2) for the storage of the medically active liquid (F) to be administered according to the present invention is located inside the housing (1). The depicted reservoir (2) is designed to be collapsible so that in the course of the emptying of the reservoir by the repeated use of the device, the soft or elastic walls deform such that the negative pressure required for withdrawing liquid from the reservoir remains substantially constant over time. A similar effect could be achieved with a rigid container that has a movable bottom by means of which the interior volume of the reservoir can also be successively be reduced (not shown).

Furthermore, the shown inhalation device comprises a pumping unit with a hollow cylinder (9) within the housing (1) which forms a pumping chamber (3) for the generation of the desired pressure which is necessary for emitting liquid (F) (i.e., the medically active liquid) and nebulising the same. The pumping unit may also comprise further components not depicted in the drawing, such as a push button, locking device, etc.

As a means for the storage of potential energy (7), a spring is provided which is coupled with one end (upwards directed, or downstream) to the cylinder (9) and which is supported at the housing (1) (lower part of the figure).

The shown inhalation device further comprises a riser pipe (5) with at least one reservoir-facing, or upstream, interior end (5A) which can be received in said cylinder (9). In other words, riser pipe (5) can be at least partially pushed into hollow cylinder (9), resulting in a decrease of the interior volume of pumping chamber (3). The term “interior volume” describes the volume of the space which extends from the reservoir-facing inlet of the cylinder (9) to the place where the interior end (5A) of the riser pipe (5) is located. In the depicted situation, riser pipe (5) is almost entirely contained in the cylinder (9). As a result, the interior volume of the pumping chamber (3), situated between inlet valve (4) and the interior end (5A) of riser pipe (5), is at a minimum.

Preferably, the section (or segment) of the hollow cylinder (9) which serves as, or accommodates, the pumping chamber (3) and which receives the riser pipe (5) exhibits a circular inner cross-section whose diameter relatively closely (e.g., except for a small gap) matches the diameter of the circular outer cross-section of the corresponding segment of the riser pipe (5). Of course, other (e.g., non-circular) cross section shapes are possible as well.

According to the depicted embodiment, inlet valve (4) is arranged between reservoir (2) and inlet of the pumping chamber (3) formed by the cylinder (9).

Furthermore, the inhalation device comprises a nozzle (6) which is connected liquid-tight to the exterior (or downstream) end (5B) of the riser pipe (5). Nozzle (6) is an impingement-type nozzle for generating the nebulised aerosol by collision of at least two liquid jets. Preferably, the cross sections of the liquid-containing channels are relatively small, typically in the region of microns.

Also depicted is an optional outlet valve (8) inside the riser pipe (5) for avoiding a backflow of liquid or air into the exterior end (5B) of the same from the outside. Outlet valve (8) is arranged in the interior end (5A) of riser pipe (5). Liquid (F) can pass outlet valve (8) in direction of nozzle (6), but outlet valve (8) blocks any undesired backflow in the opposite direction.

As can be seen in FIG. 1, riser pipe (5) is designed immobile with respect to the housing (1), and firmly attached to housing (1), indicated by the connection in the region of exterior end (5B) with housing (1). Riser pipe (5) is also firmly attached to nozzle (6), which, in turn, is attached to housing (1) as well. In contrast, the hollow cylinder (9) providing the pumping chamber (3) is designed to be moveable with respect to housing (1) and nozzle (6). The benefits of this design have been explained; reference is made to the respective sections of the description above.

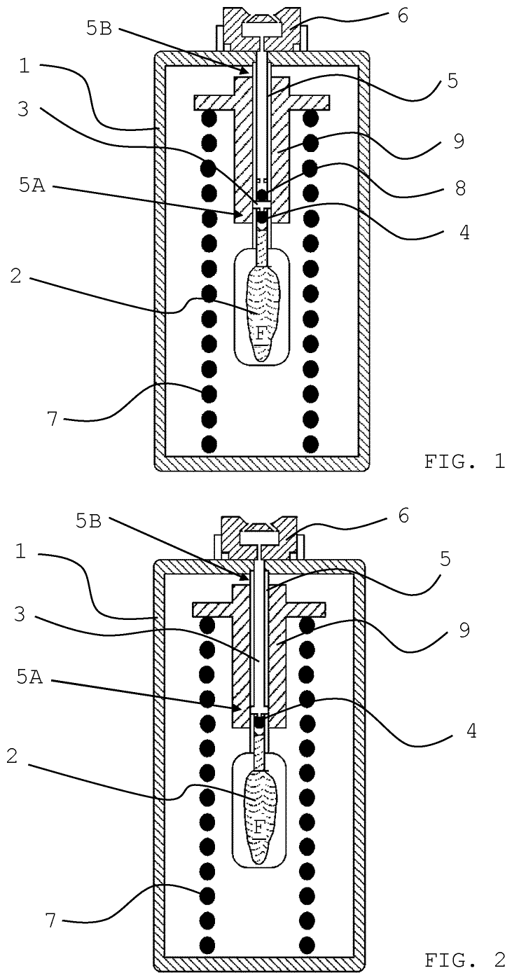

Referring to FIG. 2, a device similar to the one of FIG. 1 is depicted. However, the embodiment shown in FIG. 2 lacks the (optional) outlet valve (8). All other components are present, and also the function is comparable. In this embodiment, pumping chamber (3) extends from downstream of the valve (4) up to nozzle (6), which is the location where the fluidic resistance increases significantly. In an alternative embodiment having a particularly small inner diameter of riser pipe (5), pumping chamber (3) extends only from downstream of the valve (4) up to upstream interior end (5A) of riser pipe (5).

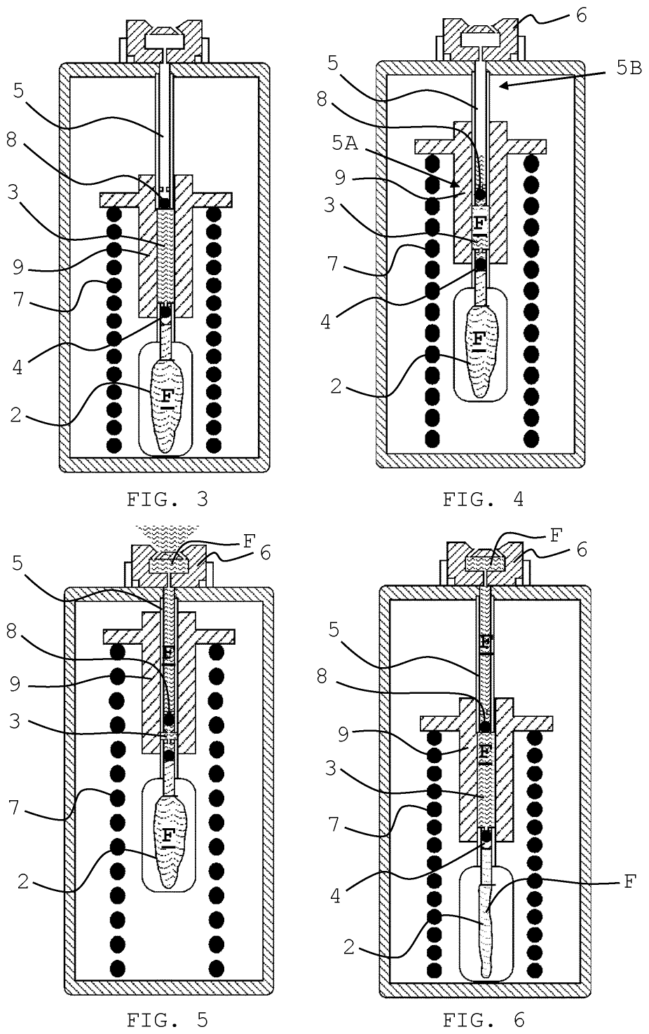

FIG. 3 shows the embodiment of FIG. 1 with a filled pumping chamber. The hollow cylinder (9) has been moved to its most upstream position, thereby loading the means for the storage of potential energy (7). Outlet valve (8) is closed due to negative pressure inside pumping chamber (3), and the inlet valve (4) is open towards the fluid reservoir (2). Increasingly collapsing walls of reservoir (2) allow the internal pressure in the reservoir (2) to remain nearly constant, while the pressure inside the pumping chamber (3) drops because of the propulsive longitudinal motion of the hollow cylinder (9), thus increasing the volume of pumping chamber (3). As a result, the pumping chamber (3) has been filled with the medically active liquid (F) from the reservoir (2).

In FIG. 4, the situation after the first actuation of the inhalation device of FIG. 1 is shown. The means for the storage of potential energy (7) has been released from the loaded position as shown in FIG. 3. It pushes the cylinder (9) in a downstream direction such as to slide over the riser pipe (5). The interior end (5A) of the riser pipe (5) has come closer to the inlet check valve (4) which is now closed. As a result, the pressure inside the pumping chamber (3) rises and keeps the inlet valve (4) closed but opens outlet valve (8). Liquid (F) flows from the riser pipe (5) through its exterior end (5B) towards nozzle (6).

FIG. 5 shows the inhalation device of FIG. 1 in the situation at the end of the aerosol emission phase. The means for the storage of potential energy (7) is in its most relaxed end position (spring fully extended). Also, the hollow cylinder (9) has been pushed almost entirely onto riser pipe (5) such that the interior volume of pumping chamber (3) has reached its minimum. Most of the liquid (F) previously contained in the pumping chamber (3) has passed outlet valve (8) into the main segment of the riser pipe (5). Some liquid (F) has been pushed towards, and though, nozzle (6), where nebulisation takes place, such that a nebulised aerosol is emitted towards the user or patient.

In FIG. 6, the inhalation device of FIG. 1 in the situation after re-filling the pumping chamber is depicted. The hollow cylinder (9) has been moved (repulsively) in an upstream direction, thus increasing the volume of the pumping chamber (3) provided by the cylinder (9). The means for the storage of potential energy (7) has been loaded (spring compressed). During movement of cylinder (9) away from the nozzle (6), a negative pressure has been generated in the pumping chamber (3), closing outlet valve (8) and opening the inlet check valve (4). As a result, further liquid (F) is drawn from reservoir (2) into the pumping chamber (3). The inhalation device's pumping chamber (3) is filled again and ready for the next ejection of liquid (F) by releasing the spring.

### LIST OF REFERENCES

1 Housing
2 Fluid reservoir, reservoir
3 Pumping chamber
4 Inlet valve
5 Riser pipe
5A Interior end
5B Exterior end
6 Nozzle
7 Means for storing potential energy, means
8 Outlet valve
9 Hollow cylinder, cylinder
F Liquid, fluid, medically active liquid
   The following examples serve to illustrate the invention, however, should not be understood as restricting the scope of the invention in any respect:

### EXAMPLES

### Materials and Methods:

Solutions of various NLRP3 inhibitors are prepared in various solvent systems (vehicles) and at various concentrations as depicted in Table 1.

Solutions of NLRP3 inhibitors 1 to 5 as summarized in Table 1 are prepared by dissolving the corresponding NLRP3 inhibitor in the chosen solvent at room temperature. If necessary, the initially generated mixtures are heated to achieve complete or highest possible solution of the corresponding NLRP3 inhibitor and then allowed to cool to room temperature.

Solutions 1 to 5 are aerosolized using a soft-mist inhaler with a working pressure of at least 200 bar and a spray duration between 1 and 2 s (seconds) as disclosed herein. Particle size distributions of the dispensed solutions are measured using a Malvern Spraytec® instrument.

TABLE 1
Solutions for particle size measurements

| Solution | NLRP3 | Mol. | Vehicle | Actual conc. |
|---|---|---|---|---|
| number | inhibitor | Weight | (vol %) | (mg/ml) |
| 1 | MCC950 sodium | 426.46 | Water 100% | 85 |
| 2 | OLT1177 | 133.17 | Water 100% | 100 |
| 3 | Bay 11-7082 | 207.25 | Ethanol 100% | 10 |
| 4 | VX-765 | 509 | Ethanol 100% | 100 |
| 5 | Parthenolide | 248.32 | Ethanol 100% | 50 |

### Example 1

Solution 1 containing 85 mg/ml of MCC950 sodium in 100% ethanol is dispensed using an embodiment of a soft mist inhaler as disclosed herein at room temperature. The particle size distribution of the resulting dispensed aerosol is expected to have a maximum below 5 μm which allows for good inhalability of the nebulized medically active liquid into the lungs of a subject.

### Example 2

Solution 2 containing 100 mg/ml of OLT1177 in pure water is dispensed using an embodiment of a soft mist inhaler as disclosed herein at room temperature. The particle size distribution of the resulting dispensed aerosol is expected to have a maximum below 5 μm which allows for good inhalability of the nebulized medically active liquid into the lungs of a subject.

### Example 3

Solution 3 containing 10 mg/ml of Bay 11-7082 in 100% ethanol is dispensed using an embodiment of a soft mist inhaler as disclosed herein at room temperature. The particle size distribution of the resulting dispensed aerosol is expected to have a maximum below 5 μm which allows for good inhalability of the nebulized medically active liquid into the lungs of a subject.

The following is a list of exemplary and non-limiting embodiments E1 to E29 comprised by the present invention:
E1. A method for the treatment or prevention of a NLRP3-associated disease, disorder or condition in a subject, the method comprising the step of administering to said subject a medically active liquid in nebulized form by inhalation,
   wherein the medically active liquid comprises a NLRP3 inhibitor and wherein the medically active liquid is administered in nebulized form using an inhalation device.
E2. The method according to embodiment E1, wherein the NLRP3-associated disease, disorder or condition is one which is responsive to inhibition of activation of the NLRP3 inflammasome.
E3. The method according to embodiment E1 or E2, wherein the NLRP3-associated disease, disorder or condition is a disease, disorder or condition of the immune system; an inflammatory disease, disorder or condition; an autoimmune disease, disorder or condition; a disease, disorder or condition of the cardiovascular system; a cancer; a tumor or other malignancy; a disease, disorder or condition of the renal system; a disease, disorder or condition of the gastro-intestinal tract; a disease, disorder or condition of the respiratory system; a disease, disorder or condition of the endocrine system; and/or a disease, disorder or condition of the central nervous system (CNS).
E4. The method according to any one of embodiments E1 to E3, wherein the NLRP3-associated disease, disorder or condition is an inflammatory disease, disorder or condition.
E5. The method according to any one of embodiments E1 to E4, wherein the NLRP3-associated disease, disorder or condition is caused by, or is associated with, a pathogen.
E6. The method according to embodiment E5, wherein the pathogen is selected from the group consisting of a virus, a bacterium, a protist, a worm, a fungus and other organisms capable of infecting a mammal.
E7. The method according to any one of embodiments E1 to E6, wherein the NLRP3-associated disease, disorder or condition is a viral infection or a disease, disorder or condition resulting from a viral infection.
E8. The method according to embodiment E7, wherein the viral infection is a coronavirus infection (e.g., SARS-CoV or SARS-CoV-2 infection).
E9. The method according to any one of embodiments E1 to E8, wherein the NLRP3-associated disease or condition is a pulmonary disease or condition.
E10. The method according to any one of embodiments E1 to E9, wherein the pulmonary disease or condition is a lower respiratory tract infection (e.g., a pneumonia).
E11. The method according to any one of embodiments E1 to E10, wherein the NLRP3-associated disease or condition is a severe acute respiratory syndrome (SARS).
E12. The method according to any one of embodiments E1 to E11, wherein the NLRP3-associated disease or condition is a SARS-CoV-2 virus infection.
E13. The method according to any one of embodiments E1 to E12, wherein the subject is a human or animal.
E14. The method according to any one of embodiments E1 to E13, wherein the subject is diagnosed with a virus infection.
E15. The method according to embodiment E14, wherein the subject is diagnosed with COVID-19.
E16. The method according to any one of embodiments E1 to E15, wherein the NLRP3 inhibitor is an inhalable NLRP3 inhibitor.
E17. The method according to any one of embodiments E1 to E16, wherein the NLRP3 inhibitor is administered to the lungs of the subject.
E18. The method according to any one of embodiments E1 to E17, wherein the NLRP3 inhibitor is a NLRP3 inflammasome inhibitor.
E19. The method according to embodiment E18, wherein the NLRP3-inhibitor inhibits NLRP3 inflammasome formation.
E20. The method according to embodiment E18, wherein the NLRP3 inhibitor inhibits NLRP3 inflammasome activation.
E21. The method according to any one of embodiments E1 to E20, wherein the NLRP3 inhibitor is a direct inhibitor of the NLRP3 protein.
E22. The method according to any one of embodiments E1 to E20, wherein the NLRP3 inhibitor is an indirect NLRP3 inhibitor.
E23. The method according to any one of embodiments E1 to E20, wherein the NLRP3 inhibitor is an inhibitor for the constituents of NLRP3 (e.g., NLRP3, apoptosis-associated speck-like protein (ASC), procaspase-1).
E24. The method according to any one of embodiments E1 to E20, wherein the NLRP3 inhibitor is selected from the group consisting of Glyburide, 16673-34-0, JC124, 1-ethyl-5-methyl-2-phenyl-1H-benzo[d]imidazole (FC11A-2), Parthenolide, VX-740, VX-765, Bay 11-7082, β-hydroxybutyrate (BHB), sulfonylureas such as MCC950, MCC7840 MNS, CY-09, N-[3,4′-dimethoxycinnamoyl]-anthranilic acid (Tranilast), OLT1177 and Oridonin.
E25. The method according to any one of embodiments E1 to E24, wherein the medically active liquid further comprises at least one further medically active compound selected from the group consisting of caspase inhibitors, SGK1 inhibitors, and/or NLRP3 inhibitors.
E26. The method according to any one of embodiments E1 to E25, wherein the inhalation device used to administer the medically active liquid comprising a NLRP3 inhibitor is a hand-held device.
E27. The method according to any one of embodiments E1 to E26, wherein the inhalation device used to administer the medically active liquid comprising the NLRP3 inhibitor is a soft-mist-inhaler.
E28. The method according to any one of embodiments E1 to E27, wherein the inhalation device used to administer the medically active liquid comprising the NLRP3 inhibitor is a soft-mist-inhaler having at least one impingement-type nozzle.
E29. The method according to any one of embodiments E1 to E28, wherein the inhalation device used to administer the medically active liquid comprising the NLRP3 inhibitor is a hand-held inhalation device for delivering a nebulised medically active aerosol for inhalation therapy, comprising
   (a) a housing having a user-facing side;
   (b) an impingement-type nozzle for generating the nebulised aerosol by collision of at least two liquid jets, the nozzle being firmly affixed to the user-facing side of the housing such as to be immobile relative to the housing;
   (c) a fluid reservoir arranged within the housing; and
   (d) a pumping unit arranged within the housing, the pumping unit having
      an upstream end that is fluidically connected to the fluid reservoir;
      a downstream end that is fluidically connected to the nozzle;
   wherein the pumping unit is adapted for pumping fluid from the fluid reservoir to the nozzle;
   wherein the pumping unit further comprises
      (i) a riser pipe having an upstream end, wherein the riser pipe is
         adapted to function as a piston in the pumping unit, and
         firmly affixed to the user-facing side of the housing such as to be immobile relative to the housing; and
      (ii) a hollow cylinder located upstream of the riser pipe, wherein the upstream end of the riser pipe is inserted in the cylinder such that the cylinder is longitudinally movable on the riser pipe;
      (iii) a lockable means for storing potential energy when locked and for releasing the stored energy when unlocked, the means being arranged outside of, and mechanically coupled to, the cylinder such that unlocking the means results in a propulsive longitudinal movement of the cylinder towards the downstream end of the pumping unit.

1. A method for the treatment or prevention of a NLRP3-associated disease, disorder or condition in a subject, the method comprising the step of administering to said subject a medically active liquid in nebulized form by inhalation,
wherein the medically active liquid comprises a NLRP3 inhibitor and wherein the medically active liquid is administered to the subject in nebulized form by inhalation using an inhalation device.

2. The method according to claim 1, wherein the NLRP3-associated disease, disorder or condition is one which is responsive to inhibition of activation of the NLRP3 inflammasome.

3. The method according to claim 1, wherein the NLRP3-associated disease, disorder or condition is a disease, disorder or condition of the immune system; an inflammatory disease, disorder or condition; an autoimmune disease, disorder or condition; a disease, disorder or condition of the cardiovascular system; a cancer; a tumor or other malignancy; a disease, disorder or condition of the renal system; a disease, disorder or condition of the gastro-intestinal tract;
a disease, disorder or condition of the respiratory system; a disease, disorder or condition of the endocrine system; and/or a disease, disorder or condition of the central nervous system (CNS).

4. The method according to claim 1, wherein the NLRP3-associated disease, disorder or condition is an inflammatory disease, disorder or condition.

5. The a method according to claim 1, wherein the NLRP3-associated disease, disorder or condition is caused by, or is associated with, a pathogen.

6. (canceled)

7. The method according to claim 1, wherein the NLRP3-associated disease, disorder or condition is a viral infection or a disease, disorder or condition resulting from a viral infection.

8. The method according to claim 7, wherein the viral infection is a coronavirus infection (e.g., SARS-CoV or SARS-CoV-2 infection).

9. The method according to claim 1, wherein the NLRP3-associated disease or condition is a pulmonary disease or condition.

10. The method according to claim 9, wherein the pulmonary disease or condition is a lower respiratory tract infection (e.g., a pneumonia).

11. The method according to claim 1, wherein the NLRP3-associated disease or condition is a severe acute respiratory syndrome (SARS).

12. The method according to claim 1, wherein the NLRP3-associated disease or condition is a SARS-CoV-2 virus infection.

13. The method according to claim 1, wherein the subject is a human or animal.

14. The method according to claim 1, wherein the subject is diagnosed with a virus infection.

15. The method according to claim 14, wherein the subject is diagnosed with COVID-19.

16. (canceled)

17. (canceled)

18. The method according to claim 1, wherein the NLRP3 inhibitor is a NLRP3 inflammasome inhibitor.

19. (canceled)

20. (canceled)

21. (canceled)

22. (canceled)

23. (canceled)

24. The method according to claim 1, wherein the NLRP3 inhibitor is selected from the group consisting of Glyburide, 16673-34-0, JC124, 1-ethyl-5-methyl-2-phenyl-1H-benzo[d]imidazole (FC11A-2), Parthenolide, VX-740, VX-765, Bay 11-7082, b-hydroxybutyrate (BHB), sulfonylureas such as MCC950, MCC7840, MNS, CY-09, N-[3,4-dimethoxycinnamoyl]-anthranilic acid (Tranilast), OLT1177 and Oridonin.

25. (canceled)

26. The method according to claim 1, wherein the inhalation device used to administer the medically active liquid comprising a NLRP3 inhibitor is a hand-held device.

27. The method according to claim 1, wherein the inhalation device used to administer the medically active liquid comprising the NLRP3 inhibitor is a soft-mist-inhaler.

28. The method according to claim 1, wherein the inhalation device used to administer the medically active liquid comprising the NLRP3 inhibitor is a soft-mist-inhaler having at least one impingement-type nozzle.

29. The method according to claim 1, wherein the inhalation device used to administer the medically active liquid comprising the NLRP3 inhibitor is a hand-held inhalation device for delivering a nebulised medically active aerosol for inhalation therapy, comprising
(a) a housing having a user-facing side;
(b) an impingement-type nozzle for generating the nebulised aerosol by collision of at least two liquid jets, the nozzle being firmly affixed to the user-facing side of the housing such as to be immobile relative to the housing;
(c) a fluid reservoir arranged within the housing; and
(d) a pumping unit arranged within the housing, the pumping unit having
an upstream end that is fluidically connected to the fluid reservoir;
a downstream end that is fluidically connected to the nozzle;
wherein the pumping unit is adapted for pumping fluid from the fluid reservoir to the nozzle;
wherein the pumping unit further comprises
(i) a riser pipe having an upstream end, wherein the riser pipe is
adapted to function as a piston in the pumping unit, and
firmly affixed to the user-facing side of the housing such as to be immobile relative to the housing; and
(ii) a hollow cylinder located upstream of the riser pipe, wherein the upstream end of the riser pipe is inserted in the cylinder such that the cylinder is longitudinally movable on the riser pipe;
(iii) a lockable means for storing potential energy when locked and for releasing the stored energy when unlocked, the means being arranged outside of, and mechanically coupled to, the cylinder such that unlocking the means results in a propulsive longitudinal movement of the cylinder towards the downstream end of the pumping unit.
